(19) United States
(12) Patent Application Publication
(10) Pub. No.: US 2024/0165096 A1
(43) Pub. Date: May 23, 2024

(54) Anti Viral Therapy

(71) Applicant: The Queen's University of Belfast, Belfast (GB)

(72) Inventors: Ahlam Ali, Belfast (GB); David Courtney, Drumaness (GB); Ultan Power, Carryduff (GB); Ken Mills, Hillcrest (IM); Lindsay Broadbent, Dundonald (GB); Connor Bamford, Lisburn (GB); Olivier Touzelet, Belfast (GB)

(21) Appl. No.: 18/550,005

(22) PCT Filed: Mar. 18, 2022
(86) PCT No.: PCT/GB2022/050695

§ 371 Date: Sep. 11, 2023

(30) Foreign Application Priority Data
Mar. 18, 2021 (GB) 2103803.9

(51) Int. Cl. A61K 31/4545; A61K 31/685; A61P 31/14

(52) U.S. Cl. CPC A61K 31/4545; A61K 31/685; A61P 31/14

(57) ABSTRACT

The invention relates to novel compounds of formula (1), or pharmaceutically acceptable salts thereof, for use in treatment of viral infection. The invention further relates to methods of treating viral infection by administering a therapeutically effective amount of the compound of formula (1).

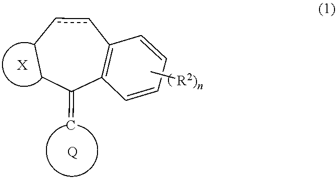

The present invention relates to the use of compounds and combined products, which include such compounds, for treating viral infection, methods of treating viral infection with such compounds and combined products, and related compositions and pharmaceutical compositions. More specifically, the compounds for use in the present invention are those of formula (1), or a pharmaceutically acceptable salt thereof. As an example, the compounds may be used for treating infection in a subject with a coronavirus, e.g. severe acute respiratory syndrome coronavirus 2 (SARS-COV-2) infection, which causes the disease known as COVID-19.

### BACKGROUND

Viral infection in human and animal populations is not a new problem. However, with the emergence of a more global society, the ability for particularly virulent infections to spread to the point of epidemic or pandemic proportions is becoming more common. In 2002-2003 the world experienced infection with Severe Acute Respiratory Syndrome

(SARS) caused by SARS-associated coronavirus (SARS-COV). In 2012 it was the turn for infection with the Middle East Respiratory Syndrome (MERS) caused by Middle East Respiratory Syndrome Coronavirus (MERS-COV).

In December 2019, the novel SARS coronavirus 2 (SARS-COV-2) was identified as the causative agent of a severe pneumonia-like coronavirus disease (COVID-19) outbreak in Wuhan in the Hubei province of China. The virus has rapidly spread throughout the world, and the World Health Organization (WHO) declared a pandemic on Mar. 11, 2020. One year later, the outbreak has resulted in more than 120 million cases globally, and caused over 2.6 million deaths. Typical COVID-19 symptoms include fever, dry cough, headache, loss of taste and/or smell and, in severe cases, pneumonia and multi-organ failure.

Emergence/re-emergence of coronaviruses, such as SARS-COV-2 in 2019, poses a serious threat to global public health, demanding the urgent development of treatments to diminish the risk of death of infected patients. The whole world was caught off guard to this challenge. To flatten the incident curve, social measures, such as quarantine, lockdown and social distancing were widely applied, leading to a sudden pause of most economic activities. Despite extensive efforts by researchers worldwide, antiviral treatment options for COVID-19, and indeed viral infection in general, are extremely limited.

Taking COVID19 as an example, only Remdesivir has thus far shown clinical evidence of an antiviral effect. The US Food & Drug Administration (FDA) has granted emergency use authorization (EUA) for Remdesivir for treatment of hospitalized COVID-19 patients. Remdesivir is a nucleotide analogue prodrug with broad antiviral activity that works as a viral RNA-dependent RNA polymerase inhibitor, originally developed to treat hepatitis C virus. It was later repurposed for Ebola virus.

The paucity of options for treating viral infections highlights the urgent and critical need to identify novel medical countermeasures both for prophylactic and therapeutic use for treating viral infection.

Certain compounds according to formula (1), as described below, are known and have also been described as having therapeutic potential. As an example, the anti-histamine Azatadine. As with Azatadine, however, no compound of formula (1) has been demonstrated to have any anti-viral activity.

### BRIEF SUMMARY OF THE DISCLOSURE

It has been surprisingly found that compounds according to formula (1) are able to treat a viral infection.

Accordingly, in a first aspect of the present invention, there is provided a compound of formula (1), or a pharmaceutically acceptable salt thereof, for use in the treatment of viral infection:

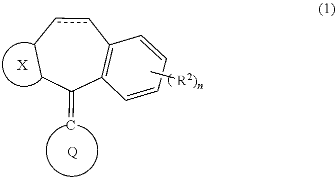

wherein:
[symbol] represents a single or a double bond;
n is an integer selected from 0 to 4;
each R2 is independently selected from halo, hydroxy, C₁₋₆ alkyl and C₁₋₆ alkoxy;
X is an optionally substituted nitrogen-containing ring selected from a 5-membered heterocycloalkyl ring, a 6-membered heterocycloalkyl ring, a 5-membered heteroaryl ring and a 6-membered heteroaryl ring; and
Q is an optionally substituted nitrogen-containing ring selected from a 5-membered heterocycloalkyl ring and a 6-membered heterocycloalkyl ring.

Such compounds have been found for the first time by the inventors to be anti-viral agents and so are useful in methods of treatment. Consequently, in a further aspect of the present invention, there is provided a method of treating viral infection in a subject, comprising administering to the subject a therapeutically effective amount of a compound according to the present invention as an anti-viral agent.

Further surprising is the finding that when administered with known anti-viral agents, such a combination provides a greater level of anti-viral activity compared to either the compounds of the present invention being administered alone, or the known anti-viral agents being administered alone. Consequently, the invention includes such novel combinations and so in a further aspect of the present invention, there is provided a composition comprising a compound of the present invention and a further anti-viral agent. As these compositions have therapeutic use, they can be prepared as pharmaceutical compositions, and so in yet a further aspect of the present invention there is provided a pharmaceutical composition comprising the composition of the earlier aspect and one or more pharmaceutically acceptable excipient.

The compound for use in treating a viral infection may therefore also be used in combination with a further anti-viral agent. The methods of the present invention may include the administration to the subject of a therapeutically effective amount of a further anti-viral agent.

The further anti-viral agent can be administered in the methods of the present invention, or for use in the context of the present invention, simultaneously, separately or sequentially with the compound of the present invention. Consequently, in a further aspect of the present invention, there is provided a combined product comprising the compound of any of claims 1 to 6 and a further anti-viral agent for simultaneous, separate or sequential use in therapy. When administered simultaneously, the compound of the present invention and the further anti-viral agent may be administered in a single composition. When administered simultaneously, the compound of the present invention and the further anti-viral agent may be administered in separate compositions.

These findings lead to the realisation that, when the compound of the present invention is used in combination with a further anti-viral agent, one can administer lesser amounts of the further anti-viral agent in order to achieve therapeutic anti-viral activity than would be expected, thereby offering the opportunity to avoid or at least reduce side-effects associated with that further anti-viral agent. Consequently, the compounds, compositions, pharmaceutical compositions, and combined products of the present invention, including those used in the methods of the present invention, can have an amount of the further anti-viral agent that is lower than the therapeutically effective amount required when the further anti-viral agent is used alone to treat viral infection. Taking Remdesivir as an example, this drug is currently prepared for administration to an adult with a body-weigh of 40 kg and above for treating viral infection with a single loading dose of 200 mg, followed by a daily maintenance dose of 100 mg once daily for 5-10 days in total. When administered simultaneously, separately or sequentially with the compounds of the present invention (e.g. Azatadine), the Remdesivir may be administered as a loading dose of less than 200 mg and/or with the following maintenance doses being less than 100 mg. Additionally, or alternatively, the number of maintenance doses may be reduced from that provided in the standard drug regimen.

As the compounds of the present invention are anti-viral agents, the compositions, pharmaceutical compositions and combined products do not require any further anti-viral agent to be effective, and so the compound of the present invention may be the only ant-viral agent provided in the compositions, pharmaceutical compositions and combined products. Similarly, the compound for use in treating viral invention and the method of the present invention may use the compound of the present invention as the only anti-viral agent. When a further anti-viral agent forms part of any aspect of the present invention, for example to take advantage of the enhancements discussed above, no further anti-viral agent may be included.

A preferred compound in each of the aspects of the present invention is Azatadine, or a pharmaceutically acceptable salt thereof. The compound may be Azatadine maleate.

Where the invention includes a further anti-viral agent, a preferred agent is Remdesivir. Consequently, a particularly preferred combination for use in all aspects of the present invention is Azatadine, or a pharmaceutically acceptable salt thereof (e.g. Azatadine maleate) and Remdesivir.

### DETAILED DESCRIPTION OF THE INVENTION

### The Compound of Formula (1)

In formula (1), X represents a nitrogen-containing ring that is fused to the 7-membered ring shown in the structural formula. Any reference to X being a 5- or 6-membered ring includes the two carbon atoms that form the fused bond with the 7-membered ring.

X represents an unsubstituted nitrogen-containing ring or a substituted nitrogen-containing ring. The substituted nitrogen-containing ring may include 1 or 2 substituents, wherein each substituent is independently selected from halo, C₁₋₆ alkyl and C₁₋₆ alkoxy, preferably halo and C₁₋₆ alkyl. The halo substituent is preferably bromo or chloro, particularly chloro.

In general, it is preferred that X represents an unsubstituted nitrogen-containing ring.

The unsubstituted or substituted nitrogen-containing ring (for X) may be selected from a 5-membered heterocycloalkyl ring, a 6-membered heterocycloalkyl ring, a 5-membered heteroaryl ring and a 6-membered heteroaryl ring. It is preferred that the nitrogen-containing ring is selected from a 6-membered heterocycloalkyl ring, a 5-membered heteroaryl ring and a 6-membered heteroaryl ring. More preferably, the nitrogen-containing ring is selected from a 5-membered heteroaryl ring and a 6-membered heteroaryl ring. Even more preferably, the nitrogen-containing ring is a 6-membered heteroaryl ring.

Generally, when X is a nitrogen-containing heterocycloalkyl or heteroaryl ring, it is preferred that the nitrogen atom is the only heteroatom in the heterocycloalkyl or heteroaryl ring.

It is preferred that X comprises or is represented by one of the following structures:

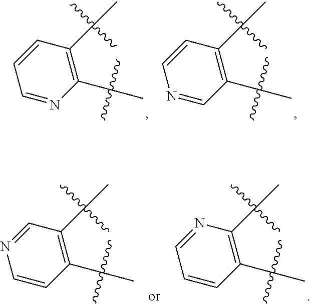 [SMILES: *c1cccnc1* ; *c1ccncc1* ; *c1ccncc1* ; *c1cccnc1*]

The wavy bonds in the structures above represent the bonds that form part of the 7-membered ring in formula (1) that are directly connected to the fused carbon-carbon bond.

Typically, the invention relates to a compound of formula (2A) or a pharmaceutically acceptable salt thereof:

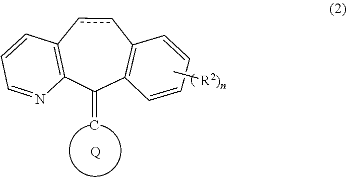

wherein Q, R² and n are as defined herein.

In formula (1) or formula (2A), Q represents a nitrogen-containing ring that is attached to a carbon-carbon double bond, as shown in the structural formulae. Any reference to Q being a 5- or 6-membered heterocycloalkyl ring includes the carbon atom that forms one end of the carbon-carbon double bond.

Q represents an unsubstituted nitrogen-containing ring or a substituted nitrogen-containing ring. The substituted nitrogen-containing ring may include one or more substituents, such as 1, 2, 3 or 4 substituents, preferably 1 to 3 substituents, more preferably 1 or 2 substituents and even more preferably one substituent.

When Q represents a substituted nitrogen-containing ring, each substituent is independently selected from halo, C₁₋₆ alkyl and C₁₋₆ alkoxy, preferably each substituent is independently selected from C₁₋₆ alkyl and C₁₋₆ alkoxy, more preferably each substituent is C₁₋₆ alkyl.

The unsubstituted or substituted nitrogen-containing ring (for Q) may be selected from 5-membered heterocycloalkyl ring and a 6-membered heterocycloalkyl ring, preferably the nitrogen-containing ring is a 6-membered heterocycloalkyl ring.

Generally, when Q is an unsubstituted nitrogen-containing ring or a substituted nitrogen-containing ring, it is preferred that the nitrogen atom is the only heteroatom in the 5- or 6-membered heterocycloalkyl ring.

It is preferred that Q comprises or is represented by one of the following structures:

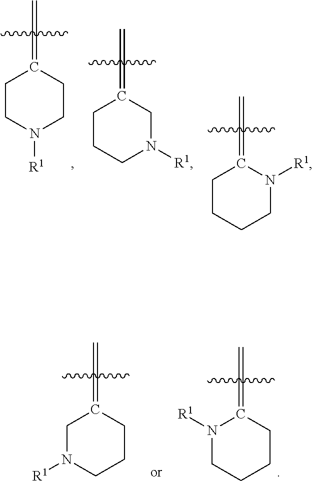

wherein R¹ is selected from C₁₋₆ alkyl and C₁₋₆ alkoxy, preferably R¹ is C₁₋₆ alkyl. It is particularly preferable for R¹ to be methyl or ethyl, preferably methyl.

The wavy bonds in the structures above represent the carbon-carbon double bond to the 7-membered ring in formula (1).

Typically, the invention relates to a compound of formula (2B), formula (3) or a pharmaceutically acceptable salt thereof:

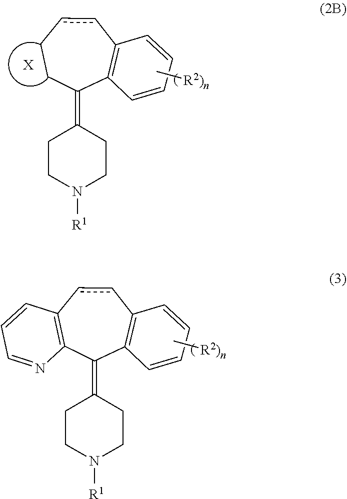

wherein X, R¹, R² and n are as defined herein.

In formulae (1), (2A), (2B) and (3), when n is an integer from 1 to 4 it represents the number of substituents (represented by R2) on the phenyl ring that is fused to the 7-membered ring.

Typically, each R² is independently selected from halo, hydroxy, C₁₋₆ alkyl and C₁₋₆ alkoxy. It is preferred that each R² is independently selected from bromo, chloro, hydroxy, C₁₋₆ alkyl and C₁₋₆ alkoxy. More preferably, each R² is independently selected from bromo, chloro, C₁₋₆ alkyl and C₁₋₆ alkoxy, particularly bromo, chloro and C₁₋₆ alkyl.

Even more preferably, each R² is independently selected from bromo and chloro, particularly chloro.

When n is not 0, it is preferred that n is an integer from 1 to 3, preferably 1 or 2. When n is 1 or 2, it is preferred that each R² on the phenyl ring is at a position meta to the 7-membered ring.

When n is 0, then there are no substituents on the phenyl ring. Generally, it is preferred that n is 0.

[symbol] represents a single or a double bond in formulae (1), (2A), (2B) and (3) above.

Generally, it is preferred that the dashed bond is a single bond.

The compound may be Azatadine, desloratidine or a pharmaceutically acceptable salt thereof. Most preferred is when the compound is azatadine or a pharmaceutically acceptable salt thereof. Azatadine has the structure shown below.

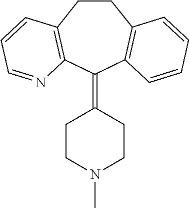 [SMILES: CN1CCC(=C2c3ccccc3CCc3cccnc32)CC1]

Compounds according to any one of formulae (1) to (3) can be prepared using the method described in U.S. Pat. No. 3,326,924 or by using other conventional methods.

The compound may be provided as a pharmaceutically-acceptable salt. The pharmaceutically acceptable salt may be convenient to purify or handle, or may be selected for its chemical compatibility with other constituents of the pharmaceutical composition.

As the compound (in formulae (1) to (3)) comprises two nitrogen-containing rings, typically it is a basic compound. A pharmaceutically acceptable salt may be formed as an addition salt from an acid having a non-toxic anion. The pharmaceutically acceptable salt of the compound may, for example, be a chloride, bromide, sulphate, phosphate, hydrogen phosphate, acetate, maleate, fumarate, lactate, tartrate, citrate, gluconate, ascorbate, aspartate, cinnamate, ethanedisulfonate, ethanesulfonate, glutamate, malate, methanesulfonate, oxalate, phenylsulfonate, propioniate, pyruvate, salicylate, stearate, succinate or toluenesulfonate salt. Of these salts the maleate salt is particularly preferred.

Most preferred is when the compound is Azatadine or a maleate salt of Azatadine.

In some instances, the compound may be acidic, and a pharmaceutically acceptable salt may be formed with a suitable cation. The pharmaceutically acceptable salt of the compound may, for example, be a sodium, potassium, calcium, magnesium or an ammonium salt.

The compounds of the present invention are used throughout all aspects of the present invention as an anti-viral agent.

### Viral Infection

The viral infection can be caused by any virus. The virus may be an RNA virus. The virus can be an influenza virus. This can be influenza A, B, C or D. Optionally the virus can be influenza A. Consequently, the virus can have a haemagglutinin content of any of H1-18. Consequently, the virus can have a neuramaninidase content of any of N1-11. The viral infection can be a Ribovirus. The viral infection can be any of Ebola virus, coronavirus, hepatitis C virus, hepatitis E virus, West Nile virus, Norovirus, Rotavirus, Poliovirus, rabies virus, measles virus, mumps virus, rhinovirus, respiratory syncytial virus, human metapneumovirus, parainfluenza virus (types 1-4) or any combination thereof. The virus may be a coronavirus. For example, any virus of the family Coronaviridae. Such viruses are characterised as enveloped viruses with a positive-sense single-stranded RNA genome and a nucleocapsid of helical symmetry. The genome size of coronaviruses may be rather large, ranging from about 26 to 32 kilobases. The virus can be SARS-COV, MERS-COV, SARS-COV-2, HCOV-299E, HCoV-OC43, HCoV-NL63, HCoV-HKU1, or any combination thereof. The virus is preferably SARS-COV-2. SARS-COV-2 is a betacoronavirus with 79% genetic homology with SARS-COV, and 98% homology to the bat coronavirus RaTG13 (Zhou et al. A pneumonia outbreak associated with a new coronavirus of probable bat origin, Nature, 2020;579 (7798):270-3). It is spread in respiratory droplets and aerosols and infects nasal, bronchial and alveolar epithelial cells by binding of the viral spike protein to its cellular receptor, ACE2 (Walls et al. Structure, Function, and Antigenicity of the SARS-COV-2 Spike Glycoprotein. Cell. 2020; 181(2):281-92 e6). Owing to the error prone nature of the viral replication process, RNA viruses such as SARS-COV-2 accumulate mutations resulting in some sequence diversity. Nevertheless, different strains of SARS-COV-2 can be recognised by sequencing and phylogenetic sequence trees. Exemplification of such phylogenetic tree analysis with rapid sequencing of isolates is reported for example by Meredith et al. Rapid Implementation of SARS-COV-2 sequencing to investigate cases of health-care associated COVID-19: a prospective genomic surveillance study in The Lancet, published on-line 14th July 2020. Sequences of amplified SARS-COV-2 virus genome can be compared with the NCBI Reference sequence NC_045512.2 or equivalent GenBank reference MN908947.3 for SARS-COV-2 corresponding to the SARS-COV-2 isolate Wuhan-Hu-1 complete genome (Wu et al. Nature 579, 265-269). SARS-COV-2 variant strains can thus be recognised and can be expected to have high level of sequence homology to the reference genome, e.g. at least 90%, at least 95%, at least 98% or at least 99%. Such sequencing surveillance can equally enable any new SARS-COV-2 virus infecting humans to be identified. The use of compounds of the present invention may therefore be used for treating humans infected with any SARS-COV virus, especially any SARS-COV-2 viral strain (which can include variants thereof).

### Further Anti-Viral Agents

The skilled person is fully aware of the range of available anti-viral therapeutics. As an example further antiviral agent may be any one or combination of the following: Idoxuridine, Trifluridine, Brivudine, Vidarabinea, Entecavir, Telbivudine, Foscarnet, Zidovudine, Didanosine, Zalcitabinea, Stavudine, Lamivudine, Abacavir, Emtricitabine, Nevirapine, Delavirdinea, Efavirenz, Etravirine, Rilpivirine, Saquinavir, Ritonavir, Indinavir, Nelfinavir, Amprenavira, Lopinavir-ritonavir, Atazanavir, Fosamprenavir, Tipranavir, Darunavir, Telaprevira, Boceprevira, Simeprevir, Asunaprevirb, Paritaprevirb, Grazoprevirb, Raltegravir, Elvitegravir, Dolutegravir, RSV-IGIVa, Palivizumab, Docosanol, Enfuvirtide, Maraviroc, VZIGa, VariZIG, Acyclovir, Ganciclovir, Famciclovir, Valacyclovir, Penciclovir, Valganciclovir, Cidofovir, Tenofovir disoproxil fumarate, Adefovir dipivoxil, Amantadinea, Ribavirin, Rimantadine, Zanamivir, Oseltamivir, Laninamivir octanoate, Peramivir, Favipiravir, Pegylated interferon alfa 2b, Interferon alfacon 1a, Fomivirsena, Podofilox, Imiquimod, Sinecatechins, Remdesivir, Flavipiravir. A preference may be for Remdesivir. Remdesivir (also known as GS-5734) is a prodrug of adenosine triphosphate and inhibits viral RNA polymerase. Remdesivir has the structure shown below.

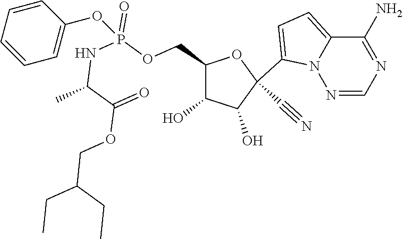 [SMILES: CCC(CC)COC(=O)[C@H](C)NP(=O)(OC[C@H]1O[C@@](C#N)(c2ccc3c(N)ncnn23)[C@H](O)[C@@H]1O)Oc1ccccc1]

A preference may be for Oseltamivir. Oseltamivir (also known as Tamiflu and GS-4104) is a neuroaminidase inhibitor and is used for treating influenza A and B. Oseltamivir has the structure shown below.

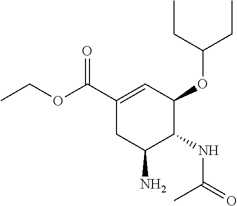 [SMILES: CCOC(=O)C1=C[C@@H](OC(CC)CC)[C@H](NC(C)=O)[C@@H](N)C1]

### Route of Administration

The compounds, composition, or combined products of the present invention may be prepared for administration intravenously, orally, nasally, mucosally by aerosolisation/nebulisation into the respiratory tract, rectally, parenterally, topically. Intravenous, oral, mucosal aerosolisation or nebulisation administration are preferred.

### Definitions

The term “anti-viral” and “anti-viral agent” relates to an ability to inhibit or prevent the progression of a viral infection, and so optionally also the damage to the host caused by such an infection. The skilled person would be aware of anti-viral activities that can achieve this goal, e.g. by termination of the virions (i.e. viricidal agents), by inhibiting the virions ability to gain entry to host cells, and/or by inhibiting the ability for virions to replicate within their host. Determining antiviral activity is well within the ordinary skill in the art, for example by measuring the capacity of a proposed anti-viral agent to diminish the cytopathic effect of a virus when administered to cells (preferably human cells) in vitro, when compared to untreated controls. There are many methods known that can quantify cytopathic effects in a group of cells, for example, CellTox™ Green Cytotoxicity Assay (Promega-UK). Any significant degree of diminution of cytopathic effect provided by the application of the proposed anti-viral agent demonstrates anti-viral activity. A more detailed example of such a study is provided below and presented in the figures. A cut off for the degree of diminution of cytopathic effect may be used to determine appropriate levels of anti-viral effect for therapeutic purposes; e.g. diminution of cytopathic effect of greater than 50%, 60%, 70%, 80% or 90%. Optionally a cut off greater than 90% may be chosen. For example, in the studies provided below, only the agent with the structure according to formula 1 satisfied this greater than 90% cut-off for human cells from the range of known anti-histamines tested.

The term “therapeutically effective amount” may be readily determined by standard methods known in the art. A “therapeutically effective amount” may at least be the least amount which ameliorates the symptoms and/or pathology of the relevant disease (e.g. viral infection) as compared to a control such as a placebo. However, more stringent requirements can define a therapeutically effective amount as the dosage approved for sale for the relevant disease (e.g. as approved by the FDA for marketing in the US).

The term “pharmaceutically acceptable excipient” in the context of the present invention would be well understood by the person skilled in the art given the specific route of administration to be used. For example, suitable excipients may include any and all solvents, dispersion media, bulking agent, coatings, antibacterial and antifungal agents, preservatives, isotonic and absorption delaying agents, and the like, compatible with pharmaceutical administration and known in the art. Except insofar as any conventional media or agent is incompatible with the active compound, use thereof in the compositions is contemplated.

The term “treatment” and “treatment of viral infection” includes both treatment of an established infection and prophylactic treatment. In the context of the treatment of viral infection, this relates to an ability to inhibit or prevent the progression of a viral infection, and so optionally also the damage to the host caused by such an infection.

The term “separately, sequentially or simultaneously” is used to define how two active agents of the present invention may be administered as part of a common therapeutic regimen. For example, the two active agents may be administered in separate dosage units, which can be administered at the same time, or sequentially. They may be administered simultaneously, as a single dosage unit, or as two separate dosage units. The route of administration may be the same for both active agents, however it is also possible that the route for each active agent is different, for example if compound 1 is administered via mucosal aerosolisation or nebulisation into the respiratory tract, while the further anti-viral agent is administered intravenously.

The term “halo” as used herein refers to a bromo (-Br), chloro (-CI), fluoro (F) or an iodo (-I) substituent. In general, it is preferred that “halo” is a bromo or chloro substituent.

The term “alkyl” as used herein refers to a straight or branched hydrocarbon chain radical consisting of carbon and hydrogen atoms, and containing no unsaturation. A “C₁₋₆ alkyl” group contains one to six carbon atoms. Unless stated otherwise specifically in the specification, an alkyl group is unsubstituted.

The term “alkoxy” as used herein refers to a radical bonded through an oxygen atom of the formula -O-alkyl, where the alkyl group is defined above. Unless stated otherwise specifically in the specification, an alkyl group is unsubstituted.

The term “heterocycloalkyl” as used herein refers to a stable non-aromatic monocyclic radical comprising or consisting of at least one carbon atom, hydrogen atoms and at least one heteroatom, wherein each heteroatom is selected from nitrogen, oxygen and sulfur. Typically, the heterocycloalkyl group comprises from one to three heteroatoms, preferably one heteroatom. When the heterocycloalkyl group is a nitrogen-containing ring, then at least one heteroatom (or where there is one heteroatom, then the only heteroatom) is nitrogen. The heterocycloalkyl group may be saturated or unsaturated, preferably is saturated. The heterocycloalkyl group is attached to the rest of the molecule by a single bond or may form part of a fused ring system. Examples of heterocycloalkyl groups include pyrrolidine, 3-pyrroline, 2-pyrroline, 2H-pyrrole, pyrazoline, imidazolidine, 2-pyrazoline, 2-imidazoline, piperidine, piperazine, morpholine and thiomorpholine.

The term “heteroaryl” as used herein refers to a stable aromatic monocyclic radical comprising or consisting of at least one carbon atom, hydrogen atoms and at least one heteroatom, wherein each heteroatom is selected from nitrogen, oxygen and sulphur. Typically, the heteroaryl group comprises from one to three heteroatoms, preferably one heteroatom. When the heteroaryl group is a nitrogen-containing ring, then at least one heteroatom (or where there is one heteroatom, then the only heteroatom) is nitrogen. The heteroaryl group is attached to the rest of the molecule by a single bond or may form part of a fused ring system. Examples of heteroaryl groups include 1H-pyrrole, pyrazole, imidazole, oxazole, isoxazole, isothiazole, thiazole, pyridine, pyridazine, pyrimidine, pyrazine and 1,2,4-triazine.

The term “nitrogen-containing ring” as used herein refers to a heterocycloalkyl ring or a heteroaryl ring comprising a nitrogen atom. The nitrogen atom may be the only heteroatom in the heterocycloalkyl or the heteroaryl ring. Alternatively, the heterocycloalkyl or the heteroaryl ring may comprise the nitrogen atom and one or more other heteroatoms, preferably wherein each heteroatom is independently selected from an oxygen atom, a sulphur atom and a nitrogen atom. It is preferred that heterocycloalkyl or the heteroaryl ring comprises two heteroatoms, wherein one of the heteroatoms is the nitrogen atom and the other heteroatom is selected from an oxygen atom, a sulphur atom and a nitrogen atom.

As used in the present disclosure, the term “comprises” has an open meaning, which allows other, unspecified features to be present. This term embraces, but is not limited to, the semi-closed term “consisting essentially of” and the closed term “consisting of”. Unless the context indicates otherwise, the term “comprises” may be replaced with either “consisting essentially of” or “consists of”. The term “consisting essentially of” may also be replaced with “consists of”.

Each document, reference, patent application or patent cited in this text is expressly incorporated herein in their entirety by reference, which means it should be read and considered by the reader as part of this text. That the document, reference, patent application or patent cited in the text is not repeated in this text is merely for reasons of conciseness. Reference to cited material or information contained in the text should not be understood as a concession that the material or information was part of the common general knowledge or was known in any country.

Aspects of the present invention will now be illustrated by way of example only and with reference to the following experimentation and to the figures.

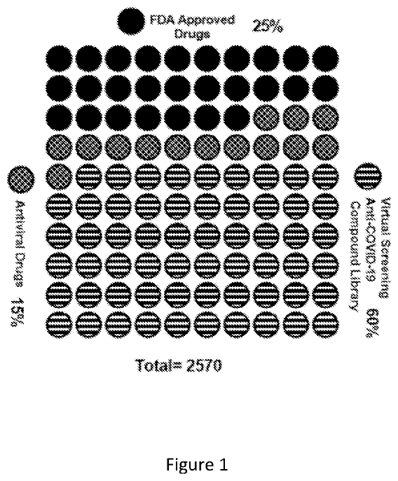

FIG. 1: Provides a visual representation of the screened library. The composition of the repurposing library included 700 FDA approved drugs, 350 Antiviral drugs, and 1,520 virtual Screening Anti-COVID-19 compounds based on 3CL protease, Spike Glycoprotein, NSP15, RDRP, PLPro and ACE2 structures.

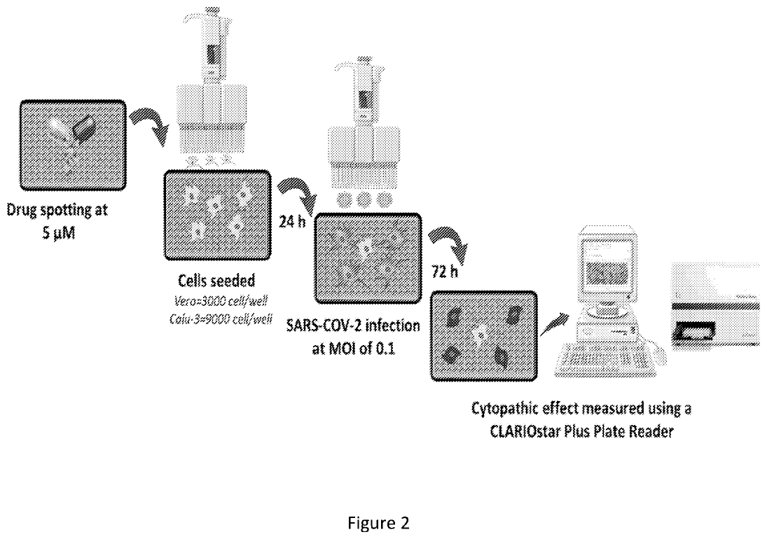

FIG. 2: Provides a visual representation of the drug screen workflow: Compounds were pre-spotted in 384-well plates at a final concentration of 5 μM, followed by cell seeding and 24 h incubation before infection with SARS-COV-2 at an MOI of 0.1. Cytopathic effect (CPE) induced by the virus was measured using CellTox™ Green Cytotoxicity Assay.

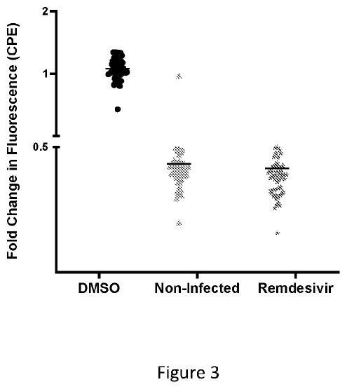

FIG. 3: Provides results of the drug screen protocol validation using 5 μM Remdesivir as a positive control and DMSO as a negative control. The graph shows Log² fold change of cytotoxicity levels after normalization to the median of each plate for all positive and negative controls as well as for non-infected cells, across all screening plates.

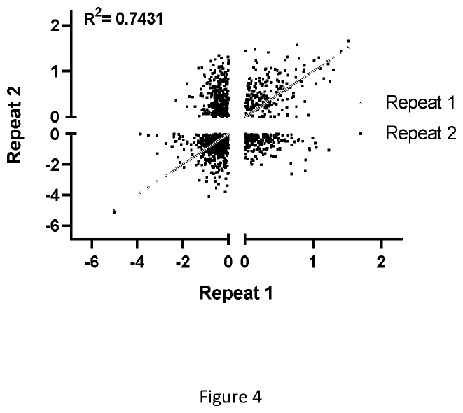

FIG. 4: Provides a correlation plot of fold change fluorescence of drug compounds in the two replicates. R² indicates the correlation coefficient for the replicates.

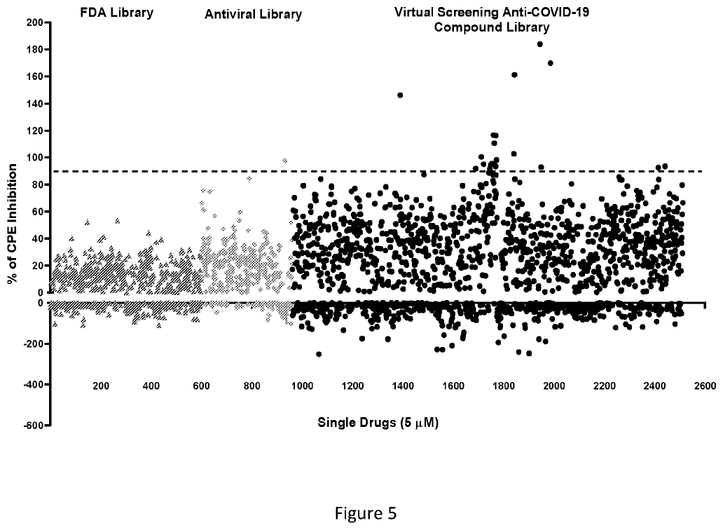

FIG. 5: Provides results of a single agent screen of 2,570 drugs across human lung cancer cell line-Calu3 cells. Drugs were added at a concentration of 5 μM and cells were incubated for 24 h, followed by SARS-COV-2 infection at MOI of 0.1. Three days post infection, the cytopathic effect was measured using CellTox™ Green reagent and Clariostar plus plate reader. Each assay plate contains one column of control wells with mock infected cells, and one column of control wells with cells infected with SAR-COV-2. All control wells were treated with DMSO at the same concentration as assay wells and used to calculate a Z′-value for each plate and to normalize the data on a per plate basis. Results were expressed as percent inhibition of CPE where 100% inhibition of CPE was equal to the mean of the mock infected cell controls, and 0% of inhibition was equal to the mean of the SAR-COV-2 infected cell controls. Each data point represents an average of two separate repeat experiments.

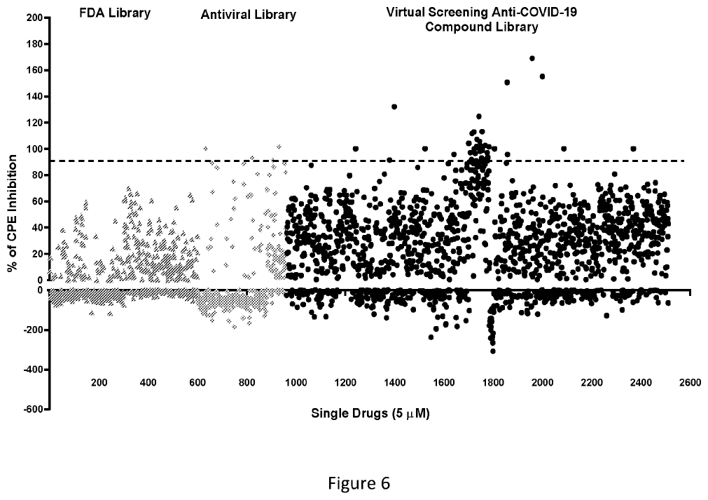

FIG. 6: Provides results of a single agent screen of 2,570 drugs across African Green Monkey cell line-Vero cells. Drugs were added at a concentration of 5 M and cells were incubated for 24 h, followed by SARS-COV-2 infection at MOI of 0.1. Three days post infection, the cytopathic effect was measured using CellTox™ Green reagent and Clariostar plus plate reader. Each assay plate contains one column of control wells with mock infected cells, and one column of control wells with cells infected with SAR-COV-2. All control wells were treated with DMSO at the same concentration as assay wells and used to calculate a Z′-value for each plate and to normalize the data on a per plate basis. Results were expressed as percent inhibition of CPE where 100% inhibition of CPE was equal to the mean of the mock infected cell controls, and 0% of inhibition was equal to the mean of the SAR-COV-2 infected cell controls. Each data point represents an average of two separate repeat experiments.

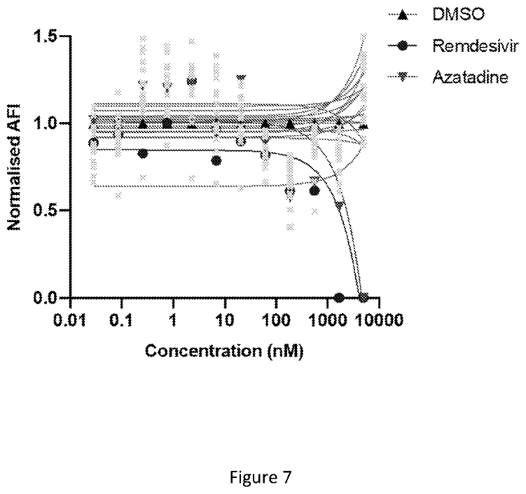

FIG. 7: Provides results of a validation study of single molecules identified in the initial drug screen using Cell Tox green. Each candidate underwent 10 serial dilutions of 1 in 3, from a starting concentration of 5 μM to test their ability to prevent cytotoxicity in SARS-COV-2 infected Calu3 cells at 72 hours post infection. Azatadine was the only candidate drug that appeared to block SARS-COV-2 mediated cytotoxicity in a comparable manner to Remdesivir.

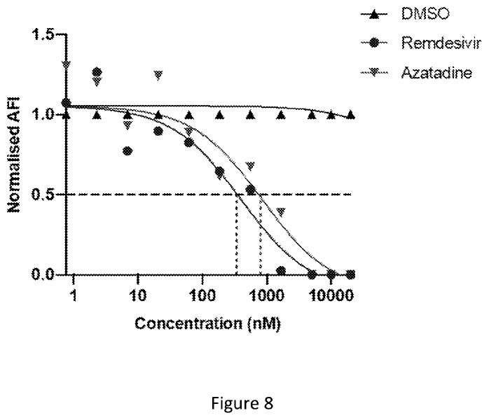

FIG. 8: Provides results of the experiment of FIG. 7 repeated in a 96 well plate format for just DMSO, Remdesivir and Azatadine and IC50s were calculated. Next Calu3 cells were treated with Azatadine at 5 and 25 μM, while DMSO was used as a negative control and Remdesivir at 1 μM as a positive control to test the molecules ability to block SARS-COV-2 replication.

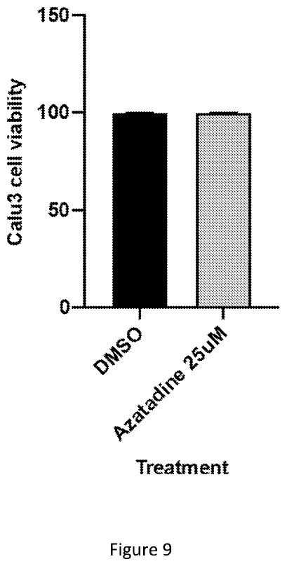

FIG. 9: Provides results of a cell viability study in response to 25 μM Azatadine assessed in Calu3 cells by CellTox™ Green with no obvious cytotoxic effects observed.

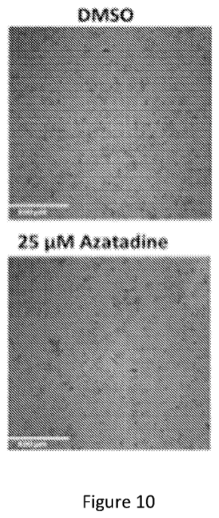

FIG. 10: Provides representative images of cells treated with DMSO or 25 μM Azatadine.

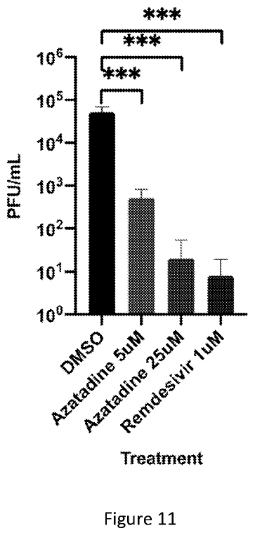

FIG. 11: Provides the results of plaque assays following the application of Azatadine and of Remdesivir. Both concentrations of Azatadine substantially reduced the levels of mature infectious viral particles released from infected cells as measured by plaque assay.

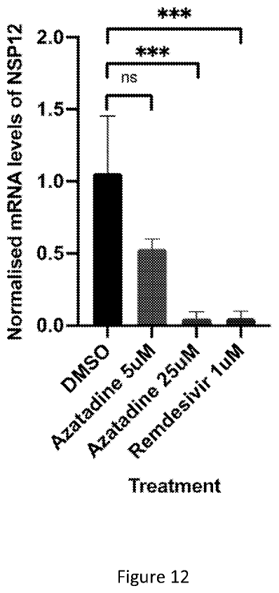

FIG. 12: Provides results of RT-qPCR analysis of mRNA levels of NSP12, following application of Azatadine and Remdesivir. Both concentrations of Azatadine substantially reduced the levels of viral NSP12 RNA as measured by RT-qPCR.

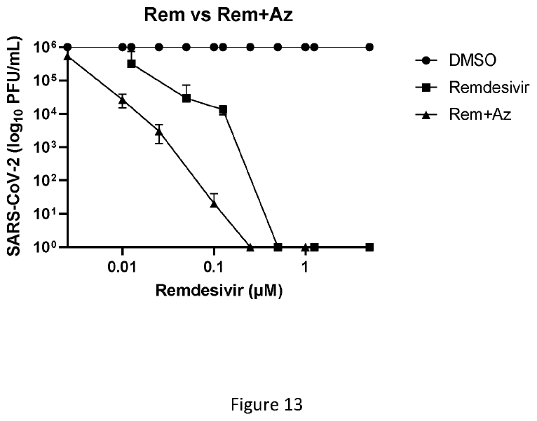

FIG. 13: Provides results of analysis of effect on viral population across a concentration gradient of Remdesivir or Remdesivir+Azatadine in combination to determine IC50s for these conditions in infected Calu3 cells.

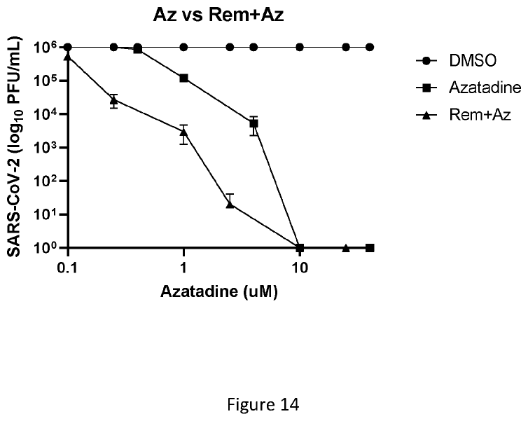

FIG. 14: Provides results of analysis of effect on viral population across concentration gradient of Azatadine or Remdesivir+Azatadine in combination to determine IC50s for these conditions in infected Calu3 cells.

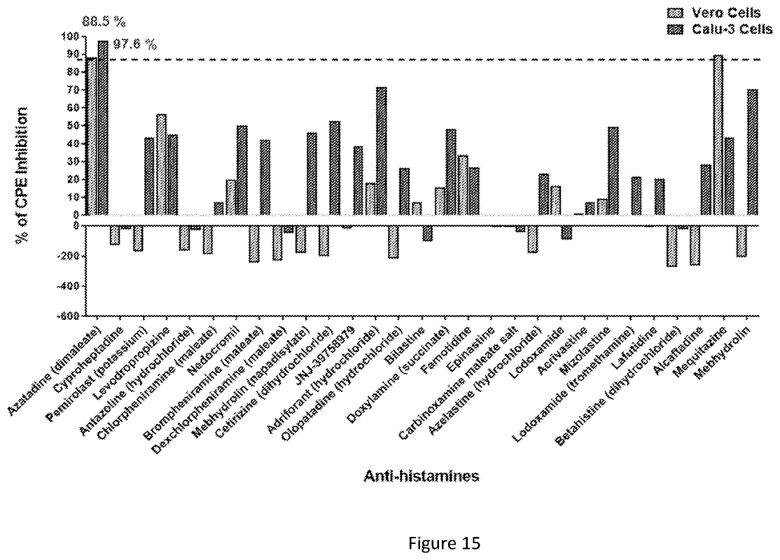

FIG. 15: Provides results of a single agent screen of 29 antihistamine drugs across the human lung cancer cell line Calu3 and Vero cells. Drugs were added at a concentration of 5 μM and cells were incubated for 24 h, followed by SARS-COV-2 infection at MOI of 0.1. Three days post infection, the cytopathic effect was measured using CellTox™ Green reagent and Clariostar plus plate reader. Each assay plate contains one column of control wells with mock-infected cells, and one column of control wells with cells infected with SAR-COV-2. All control wells were treated with DMSO at the same concentration as assay wells and used to calculate a Z′-value for each plate and to normalize the data on a per plate basis. Results were expressed as percent inhibition of CPE, where 100% inhibition of CPE was equal to the mean of the mock-infected cell controls, and 0% of inhibition was equal to the mean of the SAR-COV-2-infected cell controls. Data bars represent an average of two separate repeat experiments.

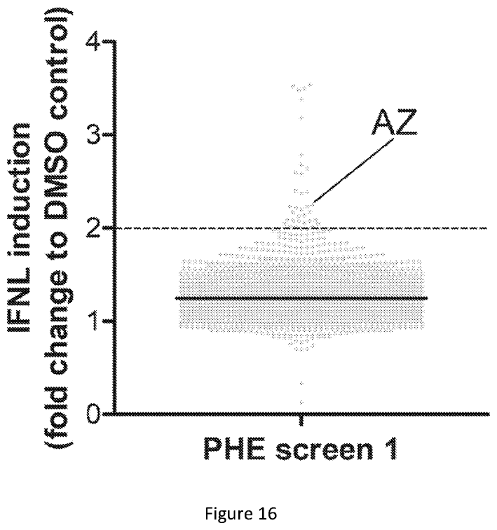

FIG. 16: Provides results of a study of induction of interferon lambda (IFNλ) by the screed drugs. Pre-treatment of Calu-3 cells with drugs for 2 h prior to infection (SARS-COV-2 PHE, moi=0.1). The release of interferon lambda (IFNλ) in Calu-3 cells at 48 hpi was detected using an IFNλ reporter cell line (HEK-293T based, expressing secreted alkaline phosphatase [SEAP] under control of an IFNλ regulated gene promoter), stimulated with non-inactivated conditioned medium (10 times dilution) for 24 h. QUANTI-Blue™, a colorimetric enzyme assay developed to determine any alkaline phosphatase (AP) activity in biological samples, was used to detect IFNλ. Following 1 h incubation, the optical density (OD) was determined at 620-655 nm using Clariostar plus plate reader.

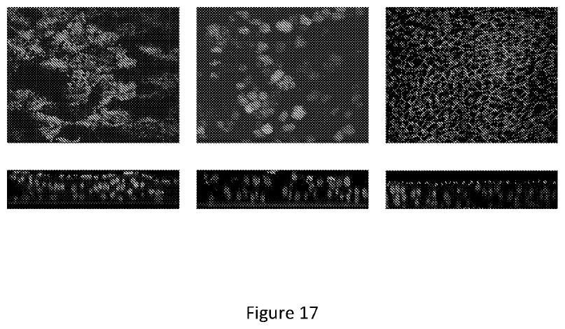

FIG. 17: Provides a confocal microscope image of the cultured cells used in this study. Representative well-differentiated human primary airway epithelial cell (WD-PAECs) cultures. WD-PAECs were grown in monolayer to ˜80% confluent then seeded onto collagen (Advanced Biomatrix) coated Transwells (6.5 mm θ, 0.4 μm pore size; Corning). Once confluent the apical medium was removed and an air-liquid interface (ALI) was initiated to trigger differentiation, which took a minimum of 21 days. Cultures are morphologically and physiologically similar to airway epithelium in vivo. Cultures were fixed with 4% (w/v) paraformaldehyde for 1 h, permeabilised with 0.2% Triton X-100 (v/v) for 1 h, blocked with 0.4% BSA (w/v) for 30 min then incubated with antibodies against β-tubulin (ciliated cells), Muc5Ac (goblet cells) or ZO-1 (tight junctions). After washing cultures were incubated with corresponding secondary antibodies (AlexaFluor). Membranes were cut from Transwells and mounted on glass slides with DAPI mounting medium (Vectashield, Vector Laboratories). Cultures were imaged on a Leica SP5 confocal microscope.

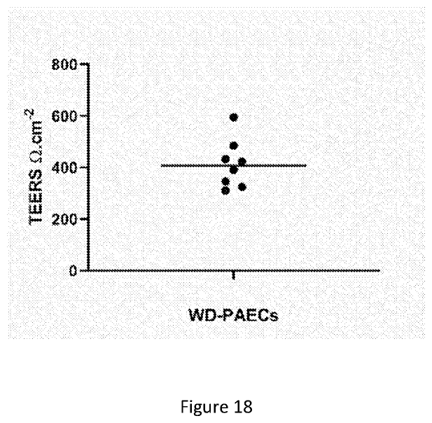

FIG. 18: Provides the results of TEER measurement of cultured cells. WD-PAECs from 8 donors were grown (as above) to complete differentiation for a minimum of 21 days. Trans-epithelial electrical resistance (TEER) was measured using an EVOM and 6 mm EndOhm chamber (World Precision Instruments). Transwells were placed in the chamber and submerged in medium. The upper electrode of the chamber was placed on top. The electrical resistance was measured across the culture. TEER >300 Ω·cm⁻² is indicative of excellent culture integrity. Representative measurements of WD-PAECs from n=8 donors.

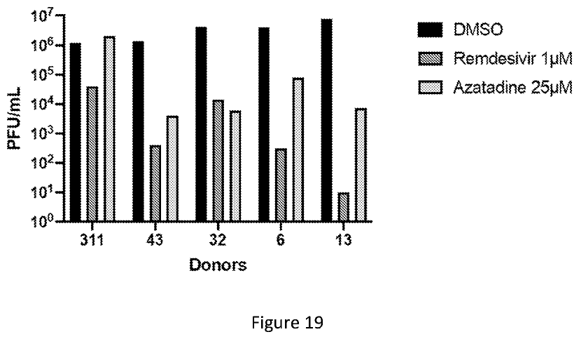

FIG. 19: Provides results of plaque assays following administration of Remdesivir or Azatadine. Cultures were infected with a low passaged clinical isolate of SARS-COV-2, BT20.1 (MOI=0.1) by the addition of 30 μL of inoculum on the apical surface for 1 h at 37ºC. Inoculum was then removed, and the apical surface was washed once with DMEM (with no additives). At 24 hpi cultures were treated with 1 μM Remdesivir, 25 μM Azatadine or DMSO as a control in both the basolateral (500 μL) and apical (10 μL) compartments. Apical washes were harvested every 24 h by the addition of 200 μL DMEM to the apical surface, incubation for 5 min and removal to a cryovial. Following washing the apical drug treatment was replaced daily. Basolateral drug treatment was replaced at 48 hpi. Apical washes at 96 hpi were titrated by plaque assay on Vero cells.

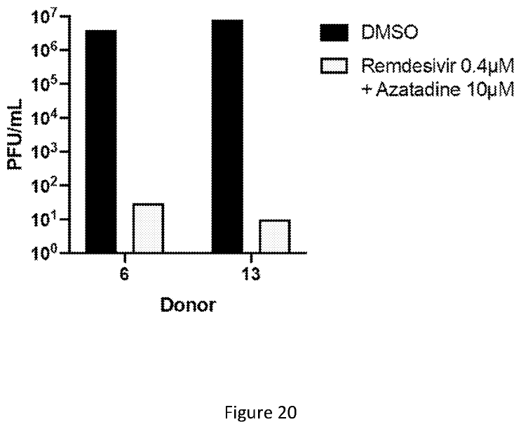

FIG. 20: Provides results of plaque assays following administration of Remdesivir and Azatadine. WD-PAEC cultures from 2 donors were treated in the same manner as above, but in this case a drug combination was used consisting of 0.4 μM Remdesivir and 10 μM Azatadine. This combination was compared to DMSO. Apical washes at 96 hpi were titrated by plaque assay on Vero cells.

A study of antiviral activity from a range of candidates was carried out as follows.

### Cell Culture

Cultures of immortalised cells were used throughout the following studies. Calu3 cells, a human lung adenocarcinoma cell line, were obtained from ATCC ((ATCC® HTB-55™) and cultured in Minimum Essential Medium α (MEM α), supplemented with 10% (v/v) foetal bovine serum, 1% (v/v) penicillin/streptomycin, 1% (v/v) L-glutamine. Vero cells (ATCC® CCL-81™), a Cercopithecus aethiops kidney cell line, as well as an isolated clone of these cells called Vero-E6 ((ATCC® CRL-1586™), were obtained from ATCC and were maintained in DMEM high glucose, pyruvate, no glutamine (Catalog number: 21969035) supplemented with 10% (v/v) foetal bovine serum, 1% (v/v) penicillin/streptomycin. All cells were maintained at 37° C. and 5% CO₂.

### SARS-CoV-2 Isolation and Growth

The SARS-COV-2 England/2/2020 (VE6-T) (EPI_ISL_407073) isolate was acquired from Public Health England, which is referred to throughout as PHE. Another isolate, V20033547, was acquired from a patient at The Royal Victoria Hospital, Belfast in June 2020 with a SARS-COV-2-speific RT-qPCR Ct value of 20. This isolate is referred to throughout as BT20.1. All SARS-COV-2 infections were performed in biosafety level 3 conditions at the Medical Biology Center biocontainment laboratory, Queen's University Belfast, and approved by the Institutional Biosafety Committee and Environmental Health and Safety. Both isolates were initially expanded on Vero-derived cells to generate viral stocks. In short, Vero E6 cells were plated in 175 cm² flasks with DMEM supplemented with 10% (v/v) foetal bovine serum, 1% (v/v) penicillin/streptomycin and allowed to attach overnight at 37° C. and 5% CO₂. Two mL of SARS-COV-2 were added to the medium (replaced to media lacking FCS and antibiotics) and the flask was then placed in a 37° C. incubator at 5% CO₂ for 3-4 days. On the final day the supernatant was harvested and centrifuged at 1,000× g for 5 min. The supernatant was aliquoted and stored at −80° C. PHE was passaged 3 additional times in total on Vero-E6 cells (to P4), the titre was determined by plaque assay and the stock was sequenced. BT20.1 was passaged 4 times in total (counting isolation) on Vero (wild-type) cells, the titre was determined by plaque assay and again the stock was sequenced.

### Drug Libraries

An in-house library of 2,570 drugs purchased from different suppliers was created. High-purity compounds (>95%) dissolved in high-quality dimethyl sulfoxide (DMSO) were used for the study (see FIG. 1). Compound quality control was performed by liquid chromatography-mass spectrometry and/or ¹H-NMR, as detailed by manufacturers. Drugs used were mostly approved drugs (FDA, EMA and other agencies).

Libraries used were SCREEN-WELL® FDA approved drug library V2 (700 compounds from Enzo, UK), Antiviral library (350 compounds from MedChemExpress, USA), and virtual Screening Anti-COVID-19 compound library based on the 3CL protease (PDB ID: 6LU7), Spike Glycoprotein (PDB ID: 6VSB), NSP15 (PDB ID: 6VVVW), RDRP, PLPro and ACE2 (Angiotensin Converting Enzyme 2) structure (1,520 compounds from MedChemExpress, USA). Libraries were prepared at 5 mM, to support a 5 μM screening format. Echo-qualified 384-well low dead volume plus microplates (LP-0200-BC; Labcyte Inc.) were used as the library source plates to support acoustic transfer with an Echo 525 Liquid Handler (Labcyte Inc.).

### SARS-CoV-2 High-Content Screening Assay

Drug compounds were acoustically transferred into black 384 well optical bottom plates (Nunc-UK). Vero cells (3×10³ cells/well) or Calu-3 cells (9×10³) were seeded in 40 μL medium. The positive control Remdesivir (MedChemExpress-USA) and the negative control DMSO were spotted on each plate. Following 24 h, plated cells were transported to the BSL3 facility where 10 μL of SARS-COV-2 diluted in assay medium were added per well at a multiplicity of infection (MOI) of 0.1. The cytopathic effect (CPE) was determined three days post infection using CellTox™ Green Cytotoxicity Assay (Promega-UK) and following the manufacturer's instructions. Plates were incubated for 15 min prior to recording fluorescence (480/520 nm) using a Synergy 2 Multi-Mode Microplate Reader (CLARIOstar Plus; BMG LABTECH, UK). See FIG. 2.

### Uninfected Host Cell Cytotoxicity Counter Screen

Compounds were acoustically transferred into black 384 well optical bottom plates (Nunc-UK). Calu-3 and Vero cells were maintained as described for the infection assay and seeded in the assay-ready plates at a density of 3×10³ cells/well for Vero cells, and 9×10³ cells/well for Calu-3 cells. Plates were incubated for 72 h at 37° C. and 5% CO₂. To assess cell toxicity, CellTox™ Green Cytotoxicity Assay (Promega-UK) was undertaken following the manufacturer's instructions. Plates were incubated for 15 min prior to recording fluorescence (480/520 nm) using a Synergy 2 Multi-Mode Microplate Reader (CLARIOstar Plus; BMG LABTECH, UK).

### Calu3 Treatment with Azatadine

Calu3 cells were seeded at a density of 5×10⁴ cell/well in a 24 well plate. Cells were seeded in the presence of Azatadine maleate, Remdesivir, DMSO or a combination of Azatadine and Remdesivir at varying concentrations. At 24 h post seeding cells were infected with the SARS-Cov-2 PHE stock at an MOI of 0.1 and incubated for 72 h. At 72 hpi supernatants were harvested from infected cells and used to determine viral titres by plaque assay. Where qPCR was performed, RNA was extracted from infected cell cultures.

### qPCR

Total RNA was isolated from infected cells using TRIzol. RNA was quantified by spectrophotometry and 1 ug was converted to cDNA using the Applied Biosciences High-Capacity cDNA Reverse Transcription Kit. A primer/probe set for detection of SARS-COV-2 NSP12 RNA was ordered from Eurofins with the sequences NSP12-Fwd: 5′-GTGARATGGTCATGTGTGGCGG-3′, NSP12-Rev: 5′-CARATGTTAAASACACTATTAGCATA-3′, and NSP12-P:5′-FAM-CAGGTGGAACCTCATCAGGAGATGC-3′. When performing qPCR the Roche probe mix for the detection of TBP (Cat. No. 05189284001) was used as a reference. The LightCycler® 480 Probes Master mix was used following the manufacturer's protocol to quantify the mRNA expression levels for all genes of interest. The ΔΔCT method was used to quantify the relative mRNA expression levels of NSP12 in infected cells.

### Plaque Assays

All SARS-COV-2 plaque assays were performed on Vero cells following the method outlined in Gordon, D.E., Jang, G.M., et al, 2020. A SARS-COV-2 protein interaction map reveals targets for drug repurposing. Nature, 583(7816), pp. 459-468. In short, Vero cells were seeded at a concentration of 7.5×10⁴ cells/well in a 24 well plate. The following day cellular supernatant containing infectious SARS-COV-2 to be quantified was serially diluted 10-fold. These dilutions were overlaid on the Vero cells for 1 h at 37° C. After adsorption 2× overlay medium in DMEM was added to give a final concentration of 2% FBS and 0.05% agarose, providing a semi-solid overlay. At 72 hpi the cells were fixed in 4% PFA for 30 min and plaques were visualised and counted using crystal violet solution.

### WD-PAEC Culture and Infection

The development of well-differentiated primary airway epithelial cell (WD-PAEC) cultures allows us to study respiratory-virus host interactions in a model that recreates the physiology and morphology of the in vivo airways. Cultures include ciliated cells, mucus producing goblet cells, non-ciliated cells, and basal cells in a pseudostratified arrangement. Primary nasal epithelial cells (n=3 donors) were obtained from consented healthy adults by brushing (Interdental Brushes, Dent-O-Care) of the nasal turbinates. Details of the donors are listed in the table 1 below. Culture and differentiation protocols have previously been describe². In brief, cells were passaged three times in Promocell Airway Epithelial Cell Growth Medium (C-21160 Promocell) then seeded onto collagen-coated Transwell supports (Corning) at 3×10⁴ cells per Transwell. After 4-6 days of submersion air-liquid interface (ALI) was initiated by removing the apical medium. Cells were differentiated using Stemcell PneumaCult ALI medium (Stemcell Technologies). Complete differentiation took a minimum of 21 days. Cultures were only used when hallmarks of excellent differentiation were evident, including extensive apical coverage with beating cilia and obvious mucus production. Tight junction formation can be assessed by measurement of the trans-epithelial electrical resistance (TEER) using an EVOM and 6 mm EndOhm chamber (World Precision Instruments). Cultures were placed in the EndOhm chamber and submerged in medium, the upper electrode of the chamber was placed on top to measure the electrical resistance through the culture in Ω. TEER readings >300 Ω·cm⁻² are indicative of intact cultures.

### Therapeutic Model of SARS-CoV-2 Infection using WD-PAEC Cultures

WD-PAECs were infected with SARS-COV-2 at a multiplicity of infection (MOI)=0.1 by addition of the inoculum on the apical surface and incubation for 1 h at 37° C. Thereafter, the inoculum was removed and the apical surface was rinsed once with DMEM (no additives). Apical washes were performed every 24 h post infection by adding 200 μL DMEM to the apical surface, incubating for 5 min, removing and storing in cryovials (Greiner) at −80° C.

TABLE 1
Demographics of donors from whom nasal epithelial cells were obtained.
Nasal Epithelial Cells

| Donor ID | Age | Sex | Race |
|---|---|---|---|
| 311 | 30 | F | Cauc |
| 43 | 31 | M | Cauc |
| 32 | 31 | M | Cauc |

At 24 hpi WD-PAECs were treated with 1 μM Remdesivir, 25 μM Azatadine or DMSO (to equate to final concentration of DMSO in Azatadine preparation). Cultures were treated with 500 μL basolaterally and 10 μL apically. Apical treatment was replaced every 24 h following washes. Basolateral treatment was replaced at 48 hpi. Titration of virus in apical washes was performed by plaque assay in Vero cells as described above.

### Statistical Analysis

For the drug screening assay, each assay plate contained one column of control wells with mock infected cells, and one column of control wells with cells infected with

SAR-COV-2. All control wells were treated with DMSO at the same concentration as assay wells and used to calculate a Z′-value for each plate and to normalize the data on a per plate basis. Results were expressed as percent inhibition of CPE, where 100% inhibition of CPE was equal to the mean of the mock infected cell controls, and 0% of inhibition was equal to the mean of the SAR-COV-2 infected cell controls.

### Results

The present study involved a high-throughput analysis of over 2,500 FDA-approved drug library to identify inhibitors of SARS-COV-2 replication in human cells (FIGS. 1-4). Candidate drugs were selected that conveyed at least 90% protection from infection of SARS-COV-2, and identified potential hits to inhibit viral replication, this included Azatadine maleate that was previously approved by the FDA as an anti-histamine (FIGS. 5 & 6). These hits were validated by determining if they had a concentration-dependent effect on protecting Calu3 cells from SARS-COV-2-induced cytopathic effects (FIG. 7). Azatadine was the only candidate that had a similarly protective profile to Remdesivir (FIG. 8). Azatadine maleate is a H1-receptor antagonist.

It antagonises the effects of histamine, which when released from the tissues causes allergic reactions. It thereby reduces the intensity of allergic reactions and tissue injury responses involving histamine release. Azatadine maleate has been shown to have anti-allergic/anti-inflammatory and mast cell-stabilizing properties. Due to its nervous system side effects, such as drowsiness, it was delisted by the FDA as it was superseded by other antihistamines, such as Loratidine, and the licence was left lapse.

In further validating the effectiveness of Azatadine maleate to inhibit SARS-COV-2 replication using standard molecular virology techniques, it was determined that Azatadine maleate does not demonstrate any toxicity on Calu3 cells at a concentration of 25 μM (FIGS. 9 & 10). Next Calu3 cells were pretreated with DMSO, Azatadine 5 μM, Azatadine 25 μM or Remdesivir 1 μM before being infected with the SARS-COV-2 PHE strain at an MOI=0.1 24 hours post treatment. Virus output from infected cells was determined by plaque assay. Azatadine 5 μM resulted in a ˜100 fold decrease in the amount of virus released from infected Calu3 cells, while Azatadine 25 μM and

Remdesivir 1 μM had a comparable inhibitory effect of ˜5000 fold (FIG. 11). Finally, we determined the viral RNA levels in these infected cells and found that Azatadine 5 μM reduced the levels of the viral NSP12 RNA by ˜50%, while Azatadine 25 μM and Remdesivir 1 μM resulted in a >95% reduction of viral NSP12 RNA levels (FIG. 12).

Remdesivir is part of the current standard of care for COVID-19. Whether Azatadine maleate could be used in combination with Remedsivir was therefore tested, and the effective concentrations of both drugs when used in combination (FIGS. 13 & 14). Calu3 cells were pre-treated with drugs 24 hours prior to infection. Cells were treated with either DMSO, Remdesivir starting at 5 μM, Azatadine starting at 40 μM, or a combination of the two with Remdesivir starting at 1 μM and Azatadine starting at 25 μM. In total 6 concentrations of each drug were used. Remdesivir alone had an IC50 of ˜0.19 μM, while

Azatadine alone had an IC50 of ˜4.9 μM. However, when used in combination, IC50s of ˜0.035 μM for Remdesivir and ˜1.3 μM for Azatadine, respectively, were calculated, which constitutes a strong reduction in drug concentrations required to inhibit viral replication. These data indicate a therapeutic synergistic effect between Remdesivir and Azatadine.

Through the comprehensive in vitro drug screen of this study it was identified that the compound Azatadine maleate has antiviral properties, against SARS-COV-2, a finding that has clinical relevance to the treatment of COVID-19. Data generated through the drug screening, assessment of viral replication and infection assays in primary ex vivo/in vitro cultures of human nasal epithelial cells in air-liquid interface further validated these candidate drugs. It should be noted that 29 other antihistamines identified in the drug library did not have antiviral activity suggesting such clinical relevance for the treatment of infection with SARS-COV-2 (FIG. 15).

The majority of available antihistamines are composed of one or two heterocyclic or aromatic rings (AR¹, AR₂) connected by nitrogen, carbon, or oxygen (X) to the ethylamine group. The nitrogen of this ethylamine group is tertiary, having two substituents (R¹, R₂). The presence of multiple aromatic or heterocyclic rings and alkyl substitutes in these antagonists results in them being lipophilic, a fact that readily explains their CNS effects. The nature of the linkage atom (X) has been used to categorize the classic antihistamines into five or six major classes.

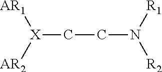

### General Formula for Antihistamines

Among all antihistamines, only Cyproheptadine shares a similar structure with Azatadine, as shown below

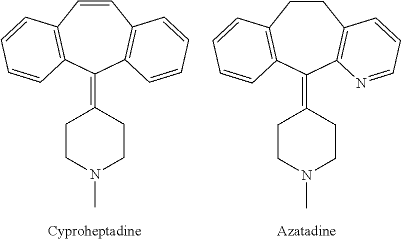

The fact that Cyproheptadine did not show any antiviral effects in our screens links the SARS-COV-2 antiviral activity of Azatadine to the tertiary amine, which is good for forming strong interactions with acidic side chains in binding clefts, also found to be active in ion channels.

The present results showed an impressive ability of Azatadine maleate to increase IFN-λ expression in SARS-COV-2-infected Calu3 cells (FIG. 16). IFN-λ has a well-established capacity to induce antiviral states in infected tissues. Recent reports demonstrated that SARS-COV-2 infection of airway epithelial cells was a relatively poor inducer of type I and III IFNs in epithelial cells. Furthermore, treatment of SARS-COV-2-infected airway or intestinal epithelial cells with IFNs significantly reduced viral replication, suggesting their therapeutic potential. Furthermore, a recent review reported evidence suggesting that type I and III IFNs may be important in controlling viraemia and modulating immune responses in COVID-19 patients. Importantly, a recent randomised, double-blind, placebo-controlled clinical trial indicated that intra-nasally nebulised IFN-B1a improved clinical outcomes in COVID-19 patients compared to controls. Thus, in addition to its direct antiviral activities, the capacity of Azatadine to enhance IFNλ responses following SARS-COV-2 infection may have important therapeutic implications.

Pre-treatment with Azatadine 2 h prior to infection (PHE, moi=0.1) enhanced the release and/or activity of IFNλ (a key antiviral cytokine) in Calu-3 cells at 48 hpi. Azatadine was in the top 1% of interferon-enhancing drugs (n=3,000). These results are based on the use of an IFNλ reporter cell line (HEK-293T based, expressing secreted alkaline phosphatase [SEAP] under control of the IFNλ promoter), stimulated with non-inactivated conditioned media (10× dilution) for 24 h. In this same experiment, Azatadine robustly inhibited SARS-COV-2-mediated cytotoxicity, consistent with previous experiments. Only one other antihistamine in the drug screen enhanced IFNλ release and/or activity (Levocetirizine Dihydrochloride) but failed to inhibit cytotoxicity and indeed actually enhanced cytotoxicity (˜1.5-fold). Other compounds enhanced IFNλ release/activity to a similar or greater extent compared to Azatadine but were not associated with a significant reduction in virus-mediated cytotoxicity, suggesting that the antiviral activity of Azatadine was not solely due to its ability to enhance IFNλ release/activity during infection.

Following infection of well characterised WD-PAEC cultures (FIGS. 17 & 18) derived from 5 donors, apically released virus was titrated in apical washes harvested at 72 hpi. All five donors demonstrated similar virus growth kinetics, reaching peak titre of between 10⁶ and 10⁷ PFU/mL (FIG. 19).

In independent experiments using WD-PAECs from the same 5 donors, cultures were infected with SARS-COV-2 (MOI=0.1). At 24 hpi cultures were treated apically and basolaterally with either remdesivir or azatadine. By 72 hpi remdesivir and azatadine treatment reduced virus growth kinetics in one donor (#43) by 10³ and 10² PFU/mL, respectively (FIG. 19). By 96 hpi four donors demonstrated a reduction in virus titres of at least 10² PFU/mL following either remdesivir or azatadine treatment, compared to the DMSO control. WD-PAECs from one donor (#311) showed no difference in SARS-COV-2 titres following azatadine treatment and was also less responsive to remdesivir, only resulting in a 1.47 log₁₀ reduction in viral titres relative to the DMSO control. The differences in response to treatment is likely donor specific.

In the same experiment cultures from two donors (#6 and #13) were also treated with a low dose combination of Remdesivir at 0.4 μM and Azatadine at 10 μM to test the synergistic inhibitory effect of the 2 drugs on SARS-COV-2 in WD-PAECs (FIG. 20). At 96 hpi both donors demonstrated a greater than 10⁵ PFU/ml reduction in virus titres.

Importantly, mast cells are major sources of cytokine release that leads to lung damage following SARS-COV-2 infection. Consequently, it has been speculated that mast cell stabilisers may also attenuate pulmonary complications, fatal inflammation and death in COVID-19. Therefore, potential beneficial effects of Azatadine maleate in COVID-19 disease, as found by these studies, are expected to be the combination of antiviral and anti-inflammatory responses in SARS-COV-2-infected patients.

### Conclusions

Azatadine maleate was not cytotoxic in Calu3 cell or Vero cells at the concentrations tested.
Azatadine maleate demonstrated strong antiviral activities in monolayer cell cultures.
Azatadine maleate demonstrated therapeutic potential against SARS-COV-2 in WD-PAEC cultures.
A combination of Azatadine maleate and Remdesivir demonstrated synergistic antiviral effects against SARS-COV-2, with significant reductions in IC50s for both drugs compared with either drug alone.
A combination of Azatadine maleate and Remdesivir demonstrated a strong antiviral effect in WD-PAEC cultures.
Azatadine maleate demonstrated a strong capacity to induce IFNλ in SARS-COV-2-infected cells, indicating its capacity to modulate immune responses to infection.

1. A compound of formula (1), or a pharmaceutically acceptable salt thereof, for use in the treatment of viral infection: 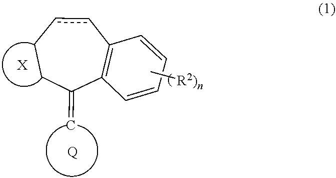
wherein: [symbol]
represents a single or a double bond;
n is an integer selected from 0 to 4;
each R² is independently selected from halo, hydroxy, C₁₋₆ alkyl and C₁₋₆ alkoxy;
X is an optionally substituted nitrogen-containing ring selected from a 5-membered heterocycloalkyl ring, a 6-membered heterocycloalkyl ring, a 5-membered heteroaryl ring and a 6-membered heteroaryl ring; and
Q is an optionally substituted nitrogen-containing ring selected from a 5-membered heterocycloalkyl ring and a 6-membered heterocycloalkyl ring.

2. The compound according to claim 1, wherein X comprises one of the following structures:

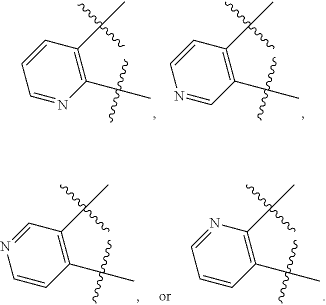 [SMILES: *c1cccnc1* ; *c1ccncc1* ; *c1ccncc1* ; *c1cccnc1*]

3. The compound according to claim 1, wherein Q comprises one of the following structures: 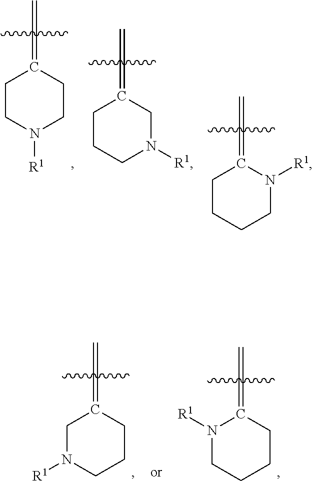
wherein R¹ is selected from C₁₋₆ alkyl and C₁₋₆ alkoxy.

4. The compound according to claim 1, wherein n is an integer from 1 to 4 and each R² is independently selected from bromo, chloro, hydroxy, C₁₋₆ alkyl and C₁₋₆ alkoxy.

5. The compound according to claim 1, wherein n is 0.

6. The compound according to claim 1, wherein the compound is Azatadine or a pharmaceutically acceptable salt thereof.

7-9. (canceled)

10. A composition comprising the compound of according to claim 1 and a second anti-viral agent.

11. The composition according to claim 10, further comprising one or more pharmaceutically acceptable excipients.

12. The composition according to claim 11, wherein the compound and the second anti-viral agent are the only anti-viral agents in the composition.

13. The composition according to claim 11, wherein the amount of the second anti-viral agent in the composition is lower than the therapeutically effective amount required for the second anti-viral agent to treat viral infection when used alone.

14. A combined product comprising the compound according to claim 1 and a econd anti-viral agent for simultaneous, separate, or sequential use in therapy.

15. (canceled)

16. The combined product according to claim 14, and the second anti-viral agent are the only anti-viral agents in the combined product.

17. (canceled)

18. A method of treating a viral infection in a subject, which comprises administering to the subject a therapeutically effective amount of a compound according to claim 1 as an anti-viral agent.

19. The method according to claim 18, wherein the compound is the only anti-viral agent used in the method.

20. The method according to claim 18, which further comprises administering to the subjecta therapeutically effective amount of a second anti-viral agent.

21. The method according to claim 18, wherein the compound and the second anti-viral agent are the only anti-viral agents used in the method.

22. The method according to claim 20, wherein the therapeutically effective amount of the second anti-viral agent is lower than the therapeutically effective amount required when the second anti-viral agent is used alone in a method of treating viral infection.

23. The composition according to claim 10, wherein the second anti-viral agent is a ribonucleotide analogue, an inhibitor of viral RNA polymerase, or Remdesivir.

24-25. (canceled)

26. The method according to claim 18, wherein the viral infection is an infection with a coronavirus.

27. The method according to claim 26, wherein the coronavirus is SARS-COV-2.
